(19) United States
(12) Patent Application Publication
(10) Pub. No.: US 2022/0061557 A1
(43) Pub. Date: Mar. 3, 2022

(54) Stress relieving attachment for bedsheets, blankets and pillowcases

(71) Applicant: Peter Cordani, Jupiter, FL (US)

(72) Inventor: Peter Cordani, Jupiter, FL (US)

(21) Appl. No.: 17/513,531

(22) Filed: Oct. 28, 2021

Related U.S. Application Data
(63) Continuation-in-part of application No. 17/395,589, filed on Aug. 6, 2021.
(60) Provisional application No. 63/061,841, filed on Aug. 6, 2020.

(51) Int. Cl. A47G 9/00; A47G 9/02

(52) U.S. Cl. CPC A47G 9/007; A47G 9/0253; A47G 9/0223; A47G 9/0238

(57) ABSTRACT

A bedsheet and/or blanket having a stress relieving squeezable element releasably secured to an edge of a bedsheet, blanket or pillow case for use in achieving stress relief, and thereby promoting restful sleep by squeezing and releasing the squeezable element. In a preferred embodiment the is place in a fabric pocket for releasable attachment to the bedsheet, blanket or pillow case by a flexible strap. The squeezable element is formed from rubber or a polymer gel.

### RELATED APPLICATIONS

In accordance with 37 C.F.R. 1.76, a claim of priority is included in an Application Data Sheet filed concurrently herewith. Accordingly, the present invention is a continuation-in-part of U.S. patent application Ser. No. 17/395,589, filed Aug. 6, 2021, entitled “Stress Relieving Insert for Bedsheets”, which claims priority to U.S. Provisional Application No. 63/061,841, filed Aug. 6, 2020, entitled “Stress Relieving Insert for Bedsheets and Blankets”, the contents of which are hereby expressly incorporated by reference.

### FIELD OF THE INVENTION

The present invention relates to bedsheets, blankets and pillowcases and, in particular, to a squeezable element such as a foam rubber insert or gel packet attached to a hem of a bedsheet, blanket, or pillowcase for use in achieving stress relief, thereby promoting restful sleep by squeezing and releasing the foam element.

### BACKGROUND OF THE INVENTION

Many people suffer with the inability to relax and fall asleep for many reasons, including the stresses of everyday life, intrusion of thoughts, and muscle and joint aches and pairis. One method of relaxing is the use of a “stress ball” which consist of a semi-soft foam rubber ball than an individual can squeeze by hand. The stress ball is proven to provide therapeutic relief from mental or physical stress by repetition of squeezing and releasing the foam rubber ball.

While the stress ball is most beneficial, the use of such a stress ball while lying in bed can be impractical. Unless the stress ball is held in a stationary position, it can be easily displaced. For instance, if the stress ball is used to help an individual fall asleep, when the individual does fall asleep the ball will be releasee from the individual's hand when sleep is bestowed upon the individual. If the individual is restless, the stress ball can be displaced by the time the individual awakens. More realistically, the individual who has a restless night may want to access the stress ball during the night. In this example, the individual may need to search for the stress ball in the darkness, wherein the ball could be entangled within the bedsheets. More critically the stress ball could fall off the bed and, should the individual need to leave the bed, the individual runs the risk on stepping upon the stress ball which could easily lead to a fall and injury. Most individuals do awaken before morning light to use a bathroom, check on strange noises, check on children, obtain water, snack and so forth. For this reason it is not recommended that a stress ball be used while the individual is in a bed. Tethering a stress ball to a bed limits movement of the ball and creates even further safety complications.

### PRIOR ART REFERENCES

U.S. Pat. No. 5,708,999 discloses an adjustable support pillow having at least one bolster portion along one edge of the support pillow, and an inflatable under-pillow. The under-pillow comprises at least one row of a plurality of parallel inflatable air chambers. Each air chamber within a row is in fluid communication with each adjacent air chamber in the same row. Each of the rows is in fluid communication with a header and the header is connectable to an air source by a nozzle.

U.S. Pat. No. 7,513,001 discloses a pillow with an attached blanket. The pillow comprises a body with one or more inwardly-turned arms. One edge of the blanket is attached to the pillow, and a pouch is formed in the pillow so that the blanket is stowable inside. A pocket may be included for holding an MP3 player or other accessory. A flap wraps around the body to cover the pouch opening and the accessory pocket when the blanket is stowed.

U.S. Pat. No. 8,234,732 discloses an orthopedic pillow having a plurality of foam layers. The foam layers include a cop foam layer made of memory foam constructed and arranged to receive a user's head. A middle foam layer is made of latex foam, the middle layer defining a central opening, and a bottom layer made of latex foam. The orthopedic pillow also lias a high density foam tube affixed to an edge of the top foam layer. The high density foam tube is constructed and arranged to support the user's neck.

U.S. 2019/0320827 discloses a sensory stress relief aid having a pillow with a plurality of different surfaces disposed on an outer layer. Each surface corresponds to a different tactile feel, such as velvet or fur. Additionally, the outer layer encloses a plurality of inner layers disposed within the pillow, wherein each inner layer comprises a different degree of firmness. A motor attached to an inflatable bladder disposed within the plurality of inner layers is configured to alternately inflate and deflate to simulate breathing. At least one speaker is included and adapted to emit soothing sounds such as nature noises and guided meditation instructions.

U.S. 10,314,413 discloses a pillow that includes a first pillow half having an outer surface and an inner surface having a first cavity formed therein filled with a first, filler material, and a second pillow half having an outer surface and an inner surface having a second cavity formed therein filled with a second filler material.

U.S. 10,292,509 discloses a pillow for seating on a base surface. The pillow includes a top surface having a negative parabolic shape or a convex shape relative to the base surface and a bottom surface having a convex shape relative to the base surface. The pillow includes up to five zones I-V, with a center zone I being designed for side sleepers, the two adjacent zones II, III being designed for back sleepers, and the two adjacent, outer zones IV, V being designed for stomach sleepers. The pillow may have different sections and parts in order to vary the feel of the pillow.

What is needed in the industry is a bedsheet, blanket, or pillow case having stress ball qualities, namely material secured to a bedsheet, blanket or pillow case than an individual may squeeze to help relieve stress while in bed. When an individual wakes up during the right and is restless, the stress relieving foam rubber insert is readily available as a recognized device for use in relieving stress to assist the individual in falling back asleep.

### SUMMARY OF THE INVENTION

Disclosed is a bedsheet, blanket or pillowcase having a semi-soft foam rubber insert attached by a strap along one hem or, alternatively, inserted into a pre-formed fabric pocket formed along one hem for use in providing a method for stress relief.

An objective of the invention is to provide a stationary stress relieving aid which can be squeezed and released by an individual, the individual concentrates on the repeated squeezing motion to alleviate stress and promote restful sleep.

Another objective of the instant invention is for the stress relieving foam rubber insert to be attached, either with a strap, or within a pre-formed fabric pocket of a bedsheet, blanket and/or pillowcase, so as to prevent loss of the stress reliever through the night.

Another objective of the instant invention is for the stress relieving foam rubber insert to be stationary, attached to a hem of a bedsheet, blanket and/or pillowcase.

Other objectives and advantages of this invention will become apparent from the following description, taken in conjunction with any accompanying drawings wherein are set forth, by way of illustration and example, certain embodiments of this invention. Any drawings contained herein constitute a part of this specification, include exemplary embodiments of. the present invention, and illustrate various objects and features thereof.

### BRIEF DESCRIPTION OF THE DRAWINGS

FIG. 1 is a perspective view of a bedsheet or blanket with a stress-relieving foam rubber insert placed within a pre-formed fabric pocket attached by a strap to the hem of the bedsheet;

FIG. 2 is a perspective view of a bedsheet or blanket with a stress-relieving foam rubber insert placed within a pre-formed fabric pocket formed along one hem of the bedsheet or blanket;

FIG. 3 is a perspective view of a pillowcase with a stress-relieving foam rubber insert placed within a pre-formed fabric pocket which is attached by a strap to the hem of the pillowcase; and

FIG. 4 is a perspective view of a pillowcase with a stress-relieving foam rubber insert placed within a pre-formed fabric pocket formed along one hem of the bedsheet or blanket.

### DESCRIPTION OF THE PREFERRED EMBODIMENT

Detailed embodiments of the instant invention are disclosed herein; however, it is to be understood that the disclosed embodiments are merely exemplary of the invention, which may be embodied in various forms. Therefore, specific functional and structural details disclosed herein are not to be interpreted as limiting, but merely as a basis for the claims and as a representation basis for teaching one skilled in the art to variously employ the present invention in virtually any appropriately detailed structure.

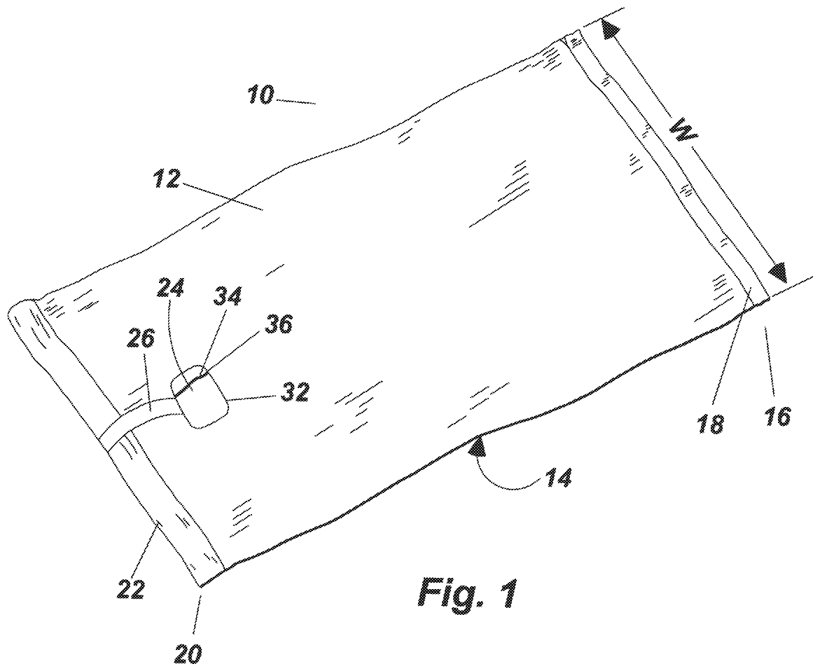

Referring to FIG. 1, disclosed is a bedsheet/blanket 10 of the instant invention defined by a piece of flat-woven textile that is used on a bed having an upper surface 12 and a lower surface 14. The upper surface 12 and the lower surface 14 are bounded along a first end 16 by a hem 18, and bounded along a second end 20 by a hem 22. In the preferred embodiment, the hem 22 includes a fabric strap 26, having a proximal end 28, sewn into the center of the second end 20 of the bedsheet/blanket 10 or releasably attached thereto by a closing member 31 such as a snap, button or hook and loop. The stress foam rubber insert 24, which is about two inches in diameter, is enclosed in a fabric pocket 32 attached to the distal end of the fabric strap 26. The fabric pocket 32 has an opening 34 having a means of closing the fabric pccket 32 around the stress foam rubber insert 24 in the manner of either a zipper or Velcro fastener 36.

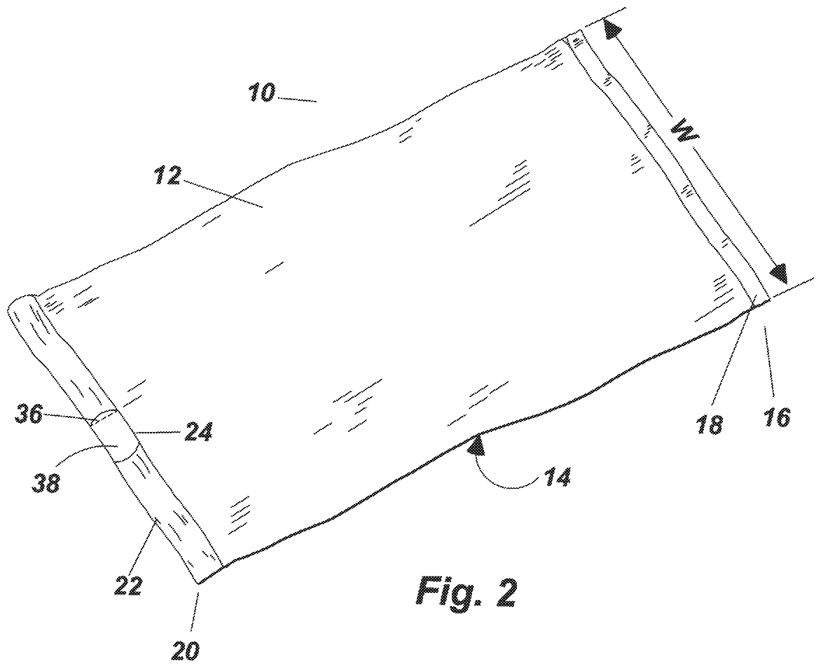

Referring to FIG. 2, disclosed is an alternate embodiment of the bedsheet/blanket 10, wherein the stress foam rubber insert 24 is enclosed in an integrated pocket 38 located in the center of the hem 22, having either a zipper or Velcro closure 36.

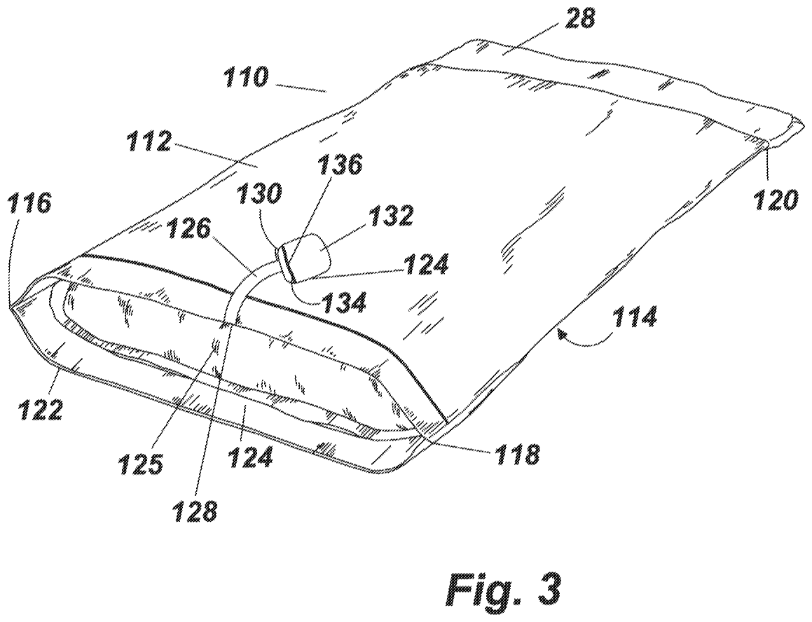

Referring to FIG. 3, disclosed is a pillowcase 110 of the instant invention defined by piece of fabric with an upper surface 112 and a lower surface 114. The upper surface 112 and the lower surface 114 are joined together by a first sewn seam 116, a second sewn seam 118, a back sewn seam 120. The pillowcase forming a front open edge 122 forming a cavity for insertion of a conventional pillow 125. In the preferred embodiment, the hem 122 includes a fabric strap 126, having a proximal end 128, sewn into the center of. the hem 122 of the pillowcase 110. The stress foam rubber insert 124, which is about, two inches in diameter, is enclosed in a fabric pocket 132 attached to the distal end 130 of the fabric strap 126. The fabric pocket 132 has an opening 134 having a means of closing 131 the fabric pocket 132 around the stress foam rubber insert 124 in the manner of either snaps, a zipper, Velcro fastener 136.

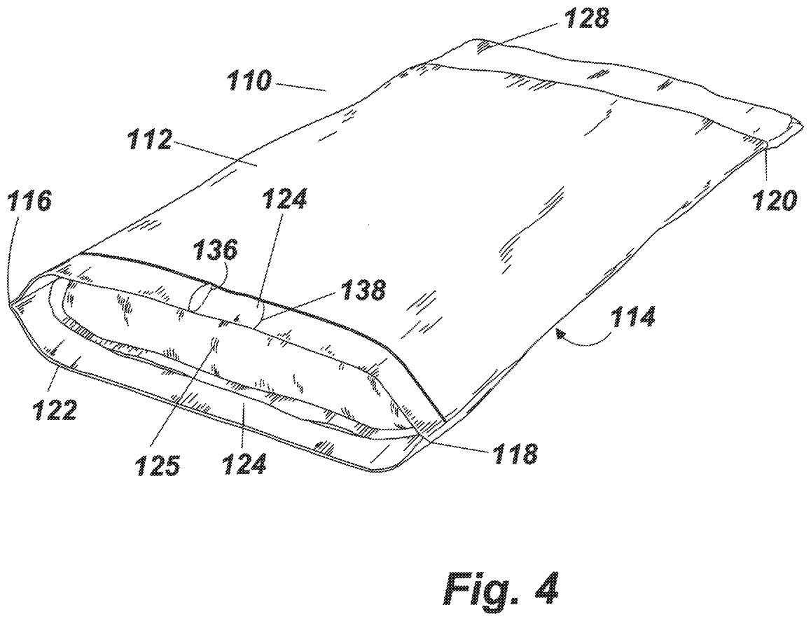

Referring to FIG. 4, disclosed is an alternate embodiment of the pillowcase 110, wherein the squeezable element is a stress foam rubber insert 124 is enclosed in an integrated pocket 138 located in the center of the hem 122, having a closure in the manner of either snaps, a zipper or Velcro }hook and loop fastener)closure 136.

The bedsheet/blanket or pillow case can be made of linen, cotton, synthetics, silk or the like. The foam rubber insert is made from a biocompatible, non-toxic material capable of providing a desired resistance. In the preferred embodiment, squeezable element is a foam rubber made of food-grade quality, natural rubber, silicone rubber, silicone and latex, an FDA-approved rubber, latex or suitable polymer, or a gel placed in a packet. A gel packet preferably contains a super absorbent polymeric and water composition. The composition utilizes a biodegradable super absorbent aqueous based polymer, namely a cross-linked modified polyacrylamides/potassium acrylate or polyacrylamides/sodium acrylate. Other suitable polymers include, albeit are not limited to, carboxy-methylcellulose, alginic acid, cross-linked starches, and cross-linked polyaminoacids.

The resistance provided by the squeezable member is similar to a stress ball, and can be measured as the force per unit area required to deform the device so that the outer circumference of the foam rubber insert can be squeezed to a point where the opposing side wails just about touch each other. The foam rubber is soft enough that laying on it would not disturb sleep, but would provide progressively sufficient resistance, wherein an elderly individual would be able to cause partial collapse of the foam rubber, and a muscular individual would be challenged to completely collapse the foam rubber. The resistance can be measured by placing the foam rubber on a scale and pressing down on the foam rubber with a one inch square surface area to determine the weight on the scale at which the top and bottom walls of the foam nearly touch each other. When the scale is calibrated in pounds, the resistance associated with the foam rubber is expressed as pounds per square inch (psi). When resistance is measured in this manner, the preferred foam rubber will have a resistance between about 20 psi and about 100 psi. Preferred resistance is between about 40 psi, but can vary based upon the diameter of the foam rubber, if round, or other shape employed. The resistance of a device depends on the size of the foam rubber, and the material from which the foam rubber is made. One of skill in the art can readily vary these parameters to produce the devices of the invention.

In use, an individual would install the bedsheet/blanket or pillow case, having the squeezable member attached by means of either a strap or an integrated enclosed pocket formed in the end of the bedsheet/blanket or pillow case. An individual can grasp or clench the squeezable member with multiple repetitions; such repetitions can be with a planned number of sets or until the individual falls asleep. Should the individual wake up during the night and become restless, the individual will easily find the stress relieving squeezable member, as it will remain in the hem of the bedsheet or blanket.

The use of the word “a” or “an” when used in conjunction with the term “comprising” in the claims and/or the specification may mean “one,” but it is also consistent with the meaning of “one or more” or “at least one.” The terra “about” means, in general, the stated value plus or minus 5%. The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs.

It is to be understood that while a certain form of the invention is illustrated, it is not to be limited to the specific form or arrangement herein described and shown. It will be apparent to those skilled in the art that various changes may be made without departing from the scope of the invention, and that the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A stress relief element secured to a bedsheet, blanket or pillow case comprising: a fabric material having at least one end forming a hem; a flexible strap having a proximal end releasably secured to said hem: a fabric pocket secured to a distal end of said strap; and a squeezable element releasably positioned in said fabric pocket.

2. The stress relief element according to claim 1 wherein said fabric pocket includes a cavity receptive to said squeezable element, said fabric pocket having an opening to said cavity with a closing element selected from the group consisting of snaps, zipper or a hook and loop fastener.

3. The stress relief element according to claim 1 wherein said squeezable element is selected from the group consisting of: closed-cell polyurethane foam rubber, natural rubber, silicone rubber, silicone and latex, or a polymer.

4. The stress relief element according to claim 1 wherein said squeezable element is a gel formed from a super absorbent polymeric material and water composition sealed in a flexible packet.

5. The stress relief element according to claim 4 wherein said super absorbent polymeric material is a biodegradable super absorbent aqueous based polymer, namely a cross-linked modified polyacrylamides/potassium acrylate or polyacrylamides/sodium acrylate.

6. The stress relief element according to claim 4 wherein said super absorbent polymeric material is a carboxy-methylcellulose, alginic acid, or cross-linked starch.

7. The stress relief element according to Claim 1 wherein said squeeze element is about two inches in diameter.

8. The stress relief element according to Claim 1 wherein said strap is releasably attached to said hem by a snap, a button or a hook and loop member.

9. The stress relief element according to Claim 1 wherein said squeezable element has a resistance of about 40 psi.
